(19) United States
(12) Patent Application Publication
(10) Pub. No.: US 2025/0161380 A1
(43) Pub. Date: May 22, 2025

(54) USE OF PROBIOTICS FOR MODULATING ALLERGIC IMMUNE RESPONSES

(71) Applicant: INTERNATIONAL N&H DENMARK APS, Kongens Lyngby (DK)

(72) Inventors: Markus LEHTINEN, Kantvik (FI); Sinikka Anneli LATVALA, Kantvik (FI); Johanna HIRVONEN, Kantvik (FI); Jenni Maria LILJAVIRTI, Kantvik (FI); Pilvi HEPO-OJA, Kantvik (FI); Neeta SENGUPTA, Kantvik (FI); Katri HOLAPPA, Kantvik (FI)

(21) Appl. No.: 18/836,135

(22) PCT Filed: Feb. 2, 2023
(86) PCT No.: PCT/EP2023/052531

§ 371 Date: Aug. 6, 2024

(30) Foreign Application Priority Data
Feb. 7, 2022 (EP) 22155334.0

(51) Int. Cl. A61K 35/747; A61K 35/744; A61P 37/06; A61P 37/08

(52) U.S. Cl. CPC A61K 35/747; A61K 35/744; A61P 37/06; A61P 37/08

(57) ABSTRACT

This invention relates to compositions comprising an effective amount of bacterial strains of the genera Lactobacillus and Lacticaseibacillus and as well as methods and uses of said compositions for the modulation of an allergic immune response in a subject.

### FIELD OF THE INVENTION

This invention relates to a new use of a composition comprising probiotic strains selected from the species Lactobacillus acidophilus and/or Lacticaseibacillus paracasei for the therapeutic modulation of an allergic immune response in a subject. The invention further relates to a method of modulation of an allergic immune response in a subject in need thereof.

### BACKGROUND OF THE INVENTION

Allergic diseases impact an estimate of 10-30% of people globally. For example, allergic rhinitis (AR) is a common disorder that affects 500 million people world-wide. Although AR is not life-threatening, it seriously impacts the quality of life and work efficiency of affected individuals and produces a large economic burden on governments. Other disease conditions that are characterized by allergic immune responses include atopic eczema, allergic asthma, food allergies, but also contact dermatitis (e.g. for latex), drug and insect bite anaphylaxis.

Allergic reactions are immunoglobulin (Ig) E mediated allergic inflammatory reactions of the mucosal or epithelial surfaces, where immune cells react to antigens and cause symptoms such as nasal/skin itching, sneezing, watery discharge, coughing and congestion.

An allergic reaction is characterized by a disrupted T-helper type 1 (Th1)/T-helper type 2 (Th2) response toward an allergen that specifically induces too strong Th2 cytokine response and causes allergic inflammation by releasing interleukins (IL)-4, IL-5, IL-9 and IL-13, inducing IgE antibody formation, promoting eosinophil development and recruitment, and increasing the production of mucus in the airways (Isolauri et al., 2001; Garn & Renz, 2007). Serum T-helper type 17 (Th17) cytokines, such as IL-17 and IL-22, are also increased for example in allergic rhinitis (AR) patients and associated with clinical symptoms, allergic inflammation, peripheral eosinophil counts, and the need for medication (Ciprandi et al. 2009; Jordakieva and Jensen-Jarolim 2018).

Circulating Th2 and Th17 cytokine levels are reduced in allergen-specific immunotherapy which is the only currently available causal treatment reducing allergy or AR-related symptoms (Jordakieva and Jensen-Jarolim 2018).

Some probiotic bacteria have been shown to modulate immune responses and to alleviate allergic symptoms. However, further evaluation of the probiotic bacteria effect and efficacy is needed for warranting the best potential efficacy for treatment of allergies.

### OBJECT OF THE INVENTION

The object of the present invention is to evaluate the effect of probiotic bacteria strains, Lactobacillus acidophilus La-14 (DGCC 11491) and Lacticaseibacillus paracasei Lpc-37 (DGCC 4981), on Th2 and Th17 cell responses of human peripheral blood mononuclear cells (PBMCs) stimulated by T-cell mitogen phytohemagglutinin (PHA).

### SUMMARY OF THE INVENTION

In one aspect, the invention relates to a therapeutic composition comprising an effective amount of one or more probiotic strains selected from the species Lactobacillus acidophilus and/or Lacticaseibacillus paracasei for use in the modulation of an allergic immune response in a subject.

In another aspect, the invention relates to a non-therapeutic use of a composition for modulation of an allergic immune response in a subject, wherein said composition comprises an effective amount of one or more bacterial strains selected from the species Lactobacillus acidophilus and/or Lacticaseibacillus paracasei.

In another aspect, the invention relates to a use of a composition comprising an effective amount of one or more probiotic strains selected from the species Lactobacillus acidophilus and/or Lacticaseibacillus paracasei for the modulation of an allergic immune response in a subject.

In a further aspect, the present invention relates to a method of modulation of an allergic immune response in a subject in need thereof, said method comprising administering to said subject a composition comprising an effective amount of one or more probiotic strains selected from the species Lactobacillus acidophilus and/or Lacticaseibacillus paracasei.

### DESCRIPTION OF FIGURES

FIG. 1. The effect of La-14 on PHA induced cytokine expression in PBMCs. The cells were treated with PHA alone or in combination with La-14.

FIG. 2. The effect of Lpc-37 on PHA induced cytokine expression in PBMCs. The cells were treated with PHA alone or in combination with Lpc-37.

FIG. 3. The effect of La-14+Lpc-37 on PHA induced cytokine expression in PBMCs. The cells were treated with PHA alone or in combination with La-14+Lpc-37.

### DETAILED DISCLOSURE OF THE INVENTION

The detailed aspects of this invention are set out below. In part some of the detailed aspects are discussed in separate sections. This is for ease of reference and is in no way limiting. All the embodiments described below are equally applicable to all aspects of the present invention unless the context specifically dictates otherwise.

### Bacteria

The bacterial strains of the present invention are selected from bacterial strains of the genera Lactobacillus and Lacticaseibacillus. Preferably the bacterial strains of the present invention are of the species Lactobacillus acidophilus and Lacticaseibacillus paracasei. In particular, the bacterial strains are chosen from Lactobacillus acidophilus strain La-14 and Lacticaseibacillus paracasei strain Lpc-37. Strains La-14 and Lpc-37 are both commercially available from DuPont Nutrition Biosciences ApS.

The bacterial strains were also deposited by DuPont Nutrition Biosciences ApS, of Langebrogade 1, DK-1411 Copenhagen K, Denmark, in accordance with the Budapest Treaty at the Leibniz-Institut Deutsche Sammlung von Mikroorganismen und Zellkulturen GmbH (DSMZ), Inhoffenstrasse 7B, 38124 Braunschweig, Germany, where they are recorded under the following registration numbers:
1. Strain La-14 (DGCC11491); deposited on 1 Jun. 2021 under registration number DSM33880.
2. Strain Lpc-37 (DGCC4981; deposited on 5 Oct. 2017 under registration number DSM32661.

Preferably the bacterial strains to be used in the present invention are bacterial strains which are generally recognised as safe and, which are preferably GRAS approved. Generally recognized as safe (GRAS) is an American Food and Drug Administration (FDA) designation that a chemical or substance added to food is considered safe by experts, and so is exempted from the usual Federal Food, Drug, and Cosmetic Act (FFDCA) food additive tolerance requirements.

### Compositions

While it is possible to administer strains of the genera Lactobacillus and Lacticaseibacillus alone according to the present invention (i.e., without any support, diluent or excipient), said strains are typically administered on or in a support as part of a product, in particular as a component or at least as one of the components of a composition, a food ingredient, a dietary supplement or a pharmaceutically acceptable composition or formulation. These products typically contain additional components well known to those skilled in the art.

Therefore, in one embodiment, the present invention relates to a therapeutic composition comprising an effective amount of one or more bacterial strain selected from the species Lactobacillus acidophilus and/or Lacticaseibacillus paracasei for use in the modulation of an allergic immune response in a subject.

In another aspect, the invention relates to a non-therapeutic use of a composition for modulation of an allergic immune response in a subject, wherein said composition comprises an effective amount of one or more bacterial strain selected from the species Lactobacillus acidophilus and/or Lacticaseibacillus paracasei.

In a further embodiment, the present invention relates to a method of modulation of an allergic immune response in a subject in need thereof, said method comprising administering to said subject a composition comprising an effective amount of one or more bacterial strain selected from the species Lactobacillus acidophilus and/or Lacticaseibacillus paracasei.

By modulation of an allergic immune response it is understood that the bacterial strains in question decrease production of Th1 and/or Th17 cytokine production from human immune cells and therefore improve Th1/Th2 balance resulting in decrease of inflammation and allergic symptoms in individuals with allergy . . . .

The term “subject”, as used herein, means a mammal.

In a particular aspect of the present invention, the subject may be a human being.

In one embodiment the subject may be female.

In one embodiment the subject may be male.

In one embodiment the subject may be with a non-binary gender.

In one embodiment the subject is not a child. The term “child” as used herein means a human being of 7 years of age or younger.

In one embodiment the subject is a human being of 8 years of age or older.

In one embodiment the subject is a human being of 16 years of age or older.

In one embodiment the subject is a human being of 18 years of age or older.

In one embodiment the subject is a healthy subject.

In a particular embodiment of the present invention, the composition comprises bacterial strains from the species Lactobacillus acidophilus and from the species Lacticaseibacillus paracasei.

In one particular embodiment, the strain of the species Lactobacillus acidophilus is strain La-14. In another particular embodiment of the present invention the strain of the species Lacticaseibacillus paracasei is strain Lpc-37.

In a particular embodiment according to the present invention, the bacterial strains of the species Lactobacillus acidophilus:Lacticaseibacillus paracasei are present in the ratio of 1:1 (v/v), preferably in the ratio 1:2, more preferably in the ration 1:3, more preferably in the ration 1:4, more preferably in the ratio 2:1, more preferably in the ratio 3:1 and more preferably in the ratio 4:1.

In another particular embodiment, the bacteria present in the composition according to the present invention are live bacteria.

In a particular embodiment of the present invention, the modulation of the allergic immune response alleviates symptoms of allergic diseases.

In another embodiment, the allergic disease is allergic rhinitis. In another embodiment, the allergic disease is atopic eczema, allergic asthma, food allergies, contact dermatitis, drug and insect bite anaphylaxis.

In a further embodiment, the composition according to the present invention is encapsulated.

The bacterial strains according to the present invention decrease the production of Th2 and immune cell derived cytokines. The Th2 and immune cell derived cytokines are IL-5 and IL-13.

In a particular embodiment, the bacterial strains of the present invention decrease the production of Th17 and immune cell derived cytokines. The Th17 and immune cell derived cytokines are IL-17 and IL-22.

The composition according to the present invention can be presented in different forms, such as a food product, food ingredient, a dietary supplement or a pharmaceutical acceptable composition or formulation.

In a particular embodiment, treatment regimen comprises oral administration of the composition.

In another particular embodiment, the treatment regimen comprises administering the composition for at least 1 week. In another embodiment, the treatment regimen comprises administering the composition for at least 2 weeks. In another embodiment, the treatment regimen comprises administering the composition at least two times per day.

### Dosage

The bacterial strains used in accordance with the present invention may be present from 10⁶ to 10¹⁴ CFU of bacteria/g of support, and more particularly from 10⁸ to 10¹² CFU of bacteria/g of support, preferably 10⁹ to 10¹² CFU/g of support.

By “support” it is meant a composition, a food product, a food ingredient, a dietary supplement or a pharmaceutically acceptable composition.

Suitably, the bacterial strains of the genera Lacticaseibacillus and Lactobacillus used in accordance with the present invention may be administered at a dosage of from about 10⁶ to about 10¹⁴ CFU of microorganism/dose, preferably from about 10⁸ to about 10¹² CFU of microorganism/dose and more preferably from about 10⁹ to about 10¹¹ CFU of microorganism/dose. CFU stands for “colony-forming units”.

By the term “per dose” it is meant that this amount of microorganism is provided to a subject either per day or per intake, preferably per day. For example, if the microorganisms are to be administered in a food product, for example in a yoghurt, then the yoghurt will preferably contain from about 10⁸ to 10¹² CFU of the microorganism. Alternatively, however, this amount of microorganism may be split into 5 multiple administrations each consisting of a smaller amount of microbial loading—so long as the overall amount of microorganism received by the subject in any specific time, for instance each 24-hour period, is from about 10⁶ to about 10¹² CFU of microorganism, preferably 10⁸ to about 10¹² CFU of microorganism and more preferably from about 10⁹ to about 10¹¹ CFU of microorganism.

In accordance with the present invention the bacterial strains are present in at least 1×10⁶ colony forming units (CFUs) per dose, preferably between 1×10⁶ and 1×10¹⁴ and more preferably between 1×10⁸ and 1×10¹². The amount of strains per dose may refer to only one of the strains or it may refer to two or more strains together.

### Food Product

In one embodiment, the bacterial strains are used according to the invention in a food product, such as a food supplement, a drink or a powder based on milk. Here, the term “food” is used in a broad sense and covers food for humans as well as food for animals (i.e. a feed). In a preferred aspect, the food is for human consumption.

The food may be in the form of a solution or as a solid, depending on the use and/or the mode of application and/or the mode of administration.

When used as, or in the preparation of, a food, such as functional food, the bacteria of the present invention may be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the bacteria of the present invention can be used as an ingredient to soft drinks, a fruit juice or a beverage comprising whey protein, health teas, cocoa drinks, milk drinks and lactic acid bacteria drinks, yoghurt and drinking yoghurt, cheese, ice cream, water ices and desserts, confectionery, biscuits cakes and cake mixes, snack foods, balanced foods and drinks, fruit fillings, care glaze, chocolate bakery filling, cheese cake flavoured filling, fruit flavoured cake filling, cake and doughnut icing, instant bakery filling creams, fillings for cookies, ready-to-use bakery filling, reduced calorie filling, adult nutritional beverage, vegetable milk, acidified soy/juice beverage, aseptic/retorted chocolate drink, bar mixes, beverage powders, calcium fortified soy/plain and chocolate milk, calcium fortified coffee beverage.

Advantageously, where the product is a food product, the bacterial strains should remain effective through the normal “sell-by” or “expiration” date during which the food product is offered for sale by the retailer. Preferably, the effective time should extend past such dates until the end of the normal freshness period when food spoilage becomes apparent. The desired lengths of time and normal shelf life will vary from foodstuff to foodstuff and those of ordinary skill in the art will recognise that shelf-life times will vary upon the type of foodstuff, the size of the foodstuff, storage temperatures, processing conditions, packaging material and packaging equipment age.

### Food Ingredients

Compositions of the present invention may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Dietary Supplements

The compositions of the present invention may take the form of dietary supplements or may themselves be used in combination with dietary supplements, also referred to herein as food supplements.

The term “dietary supplement” as used herein refers to a product intended for ingestion that contains a “dietary ingredient” intended to add nutritional value or health benefits to (supplement) the diet. A “dietary ingredient” may include (but is not limited to) one, or any combination, of the following substances: bacteria, a probiotic (e.g. probiotic bacteria), a vitamin, a mineral, a herb or other botanical, an amino acid, a dietary substance for use by people to supplement the diet by increasing the total dietary intake, a concentrate, metabolite, constituent, or extract.

Dietary supplements may be found in many forms such as tablets, capsules, soft gels, gel caps, liquids, or powders. Some dietary supplements can help ensure an adequate dietary intake of essential nutrients; others may help prevent or treat diseases.

### Medical Food

Compositions of the present invention may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### Pharmaceutical Composition

The bacteria of the present invention may be used as—or in the preparation of—a pharmaceutical composition or formulation. Here, the term “pharmaceutical” is used in a broad sense—and covers pharmaceuticals for humans as well as pharmaceuticals for animals (i.e. veterinary applications).

In a preferred embodiment, the pharmaceutical acceptable composition is a medicament.

The pharmaceutical composition can be for therapeutic purposes-which may be curative or palliative or preventative in nature.

In a preferred embodiment of the present invention, the medicament is for oral administration.

A pharmaceutically acceptable composition or support may be for example a formulation or support in the form of creams, foams, gels, lotions, and ointments of compressed tablets, tablets, capsules, ointments, suppositories, sprays, such as nasal sprays, or drinkable solutions.

When used as—or in the preparation of—a pharmaceutical, the composition of the present invention may be used in conjunction with one or more of: a pharmaceutically acceptable carrier, a pharmaceutically acceptable diluent, a pharmaceutically acceptable excipient, a pharmaceutically acceptable adjuvant, a pharmaceutically active ingredient.

The pharmaceutical may be in the form of a solution or as a solid—depending on the use and/or the mode of application and/or the mode of administration.

The bacterial strains of the present invention may be used as pharmaceutical ingredients. Here, the composition may be the sole active component, or it may be at least one of a number (i.e. 2 or more) of active components.

The bacterial strains may be used according to the present invention in any suitable form—whether when alone or when present in a combination with other components or ingredients. Likewise, combinations comprising the bacteria of the present invention and other components and/or ingredients (i.e. ingredients—such as food ingredients, functional food ingredients or pharmaceutical ingredients) may be used in any suitable form.

The bacterial strains may be used according to the present invention in the form of solid or liquid preparations or alternatives thereof. Examples of solid preparations include, but are not limited to tablets, capsules, dusts, granules and powders which may be water dispersible, spray-dried or freeze-dried. Examples of liquid preparations include, but are not limited to, aqueous, organic or aqueous-organic solutions, suspensions and emulsions.

Suitable examples of forms include one or more of: tablets, pills, capsules, ovules, solutions or suspensions, which may contain flavouring or colouring agents, for immediate-, delayed-, modified-, sustained-, pulsed- or controlled-release applications.

By way of example, if the bacteria of the present invention are used in a tablet form—such for use as a functional ingredient—the tablets may also contain one or more of: excipients such as microcrystalline cellulose, lactose, sodium citrate, calcium carbonate, dibasic calcium phosphate and glycine; disintegrants such as starch (preferably corn, potato or tapioca starch), sodium starch glycollate, croscarmellose sodium and certain complex silicates; granulation binders such polyvinylpyrrolidone, as hydroxypropylmethylcellulose (HPMC), hydroxypropylcellulose (HPC), sucrose, gelatin and acacia; lubricating agents such as magnesium stearate, stearic acid, glyceryl behenate and talc may be included.

Examples of nutritionally acceptable carriers for use in preparing the forms include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid 30 monoglycerides and diglycerides, petroethrai fatty acid esters, hydroxymethylcellulose, polyvinylpyrrolidone, and the like.

Preferred excipients for the forms include lactose, starch, a cellulose, milk sugar or high molecular weight polyethylene glycols.

For aqueous suspensions and/or elixirs, the bacteria of the present invention may be combined with various sweetening or flavouring agents, colouring matter or dyes, with emulsifying and/or suspending agents and with diluents such as water, propylene glycol and glycerin, and combinations thereof.

The forms may also include gelatine capsules; fibre capsules, fibre tablets etc.; or even fibre beverages.

In one aspect, the bacteria according to the present invention may be administered in an aerosol, for example by way of a nasal spray, for instance for administration to the respiratory tract.

### Prebiotics

In one embodiment, the bacterial strains and compositions of the present invention may further be combined or comprise one or more fibres and/or prebiotics.

Prebiotics are defined as a substrate that is selectively utilized by host microorganisms conferring a health benefit. These are generally ingredients that beneficially affect the health of the host by selectively stimulating the growth and/or activity of one or a limited number of bacteria, and thus improve host health. The prebiotic can be applied to oral route. Typically, prebiotics are carbohydrates (such as oligosaccharides), but the definition does not preclude non-carbohydrates, such as polyphenols, or polyunsaturated fatty acids or other ingredients that can be utilized selectively by a limited number of bacteria to confer a health benefit. The most prevalent forms of prebiotics are nutritionally classed as soluble fibres. To some extent, many forms of dietary fibres exhibit some level of prebiotic effect.

In one embodiment, a prebiotic is a selectively fermented ingredient that allows specific changes, both in the composition and/or activity in the gastrointestinal or skin microflora that confers benefits upon host well-being and health.

Suitably, the prebiotic may be used according to the present invention in an amount of 0.01 to 100 g/day, preferably 0.1 to 50 g/day, more preferably 0.5 to 20 g/day. In one embodiment, the prebiotic may be used according to the present invention in an amount of 1 to 10 g/day, preferably 2 to 9 g/day, more preferably 3 to 8 g/day. In another embodiment, the prebiotic may be used according to the present invention in an amount of 5 to 50 g/day, preferably 5 to 25 g/day.

Examples of dietary sources of prebiotics include soybeans, inulin sources (such as Jerusalem artichoke, jicama, and chicory root), raw oats, unrefined wheat and unrefined barley.

Examples of suitable prebiotics include alginate, xanthan, pectin, locust bean gum (LBG), inulin, guar gum, galacto-oligosaccharide (GOS), fructo-oligosaccharide (FOS), polydextrose 10 (i.e. Litesse®), lactitol, L-Arabinose, D-Xylose, L-Rhamnose, D-Mannose, L-Fucose, inositol, sorbitol, mannitol, xylitol, fructose, carrageenan, alginate, microcrystalline cellulose (MCC), betaine, lactosucrose, soybean oligosaccharides, isomaltulose (Palatinose™), isomalto-oligosaccharides, gluco-oligosaccharides, xylooligosaccharides, manno-oligosaccharides, beta-glucans, cellobiose, raffinose, gentiobiose, melibiose, xylobiose, cyciodextrins, isomaltose, trehalose, stachyose, panose, pullulan, verbascose, galactomannans, (human) milk oligosaccharides and all forms of resistant starches.

The combination of one or more of the bacterial strains according to the present invention and one or more fibres and/or prebiotics according to the present invention exhibits a synergistic effect in certain applications (i.e. an effect which is greater than the additive effect of the bacteria when used separately).

In one embodiment, the bacterial strains or a mixture thereof according to the present invention is used in combination with one or more fibres and/or prebiotic.

Suitably, the prebiotic used is polydextrose, lactitol, inositol, L-Arabinose, D-Xylose, L-Rhamnose, D-Mannose, L-Fucose, sorbitol, mannitol, xylitol, fructose, carrageenan, alginate, 5 microcrystalline cellulose (MCC), milk oligosaccharide or betaine.

In a further aspect, the invention relates to a composition, food products, food ingredient, dietary supplements or a pharmaceutical acceptable composition comprising bacterial strains according to the present invention or a mixture thereof and one or more fibres and/or a prebiotic.

### Embodiments of the Invention

For the avoidance of doubt, some of the embodiments the present invention relates to are set out below:

Embodiment 1. A therapeutic composition comprising an effective amount of one or more bacterial strains selected from the species Lactobacillus acidophilus and/or Lacticaseibacillus paracasei for use in the modulation of an allergic immune response in a subject.

Embodiment 2. A non-therapeutic use of a composition for modulation of an allergic immune response in a subject, wherein said composition comprises an effective amount of one or more bacterial strains selected from the species Lactobacillus acidophilus and/or Lacticaseibacillus paracasei.

Embodiment 3. The composition according to embodiment 1 or 2, wherein said composition comprises bacterial strains from the species Lactobacillus acidophilus and from the species Lacticaseibacillus paracasei.

Embodiment 4. The composition according to embodiments 1-3, wherein the bacterial strain of the species Lactobacillus acidophilus is strain La-14.

Embodiment 5. The composition according to embodiments 1-3, wherein the bacterial strain of the species Lacticaseibacillus paracasei is strain Lpc-37.

Embodiment 6. The composition according to any one of embodiments 1-5, wherein the bacterial strains are present in at least 1×10⁶ colony forming units (CFUs) per dose, preferably between 1×10⁶ and 1×10¹⁴ and more preferably between 1×10⁸ and 1×10¹².

Embodiment 7. The composition according to any one of embodiments 1-6, wherein said modulation of the allergic immune response alleviates symptoms of allergic diseases.

Embodiment 8. The composition according to embodiment 7, wherein the allergic disease is allergic rhinitis.

Embodiment 9. The composition according to embodiment 7, wherein the allergic disease is atopic eczema, allergic asthma, food allergies, contact dermatitis, drug and insect bite anaphylaxis.

Embodiment 10. The composition according to embodiments 1-6, wherein said bacterial strains decrease the production of Th2 and immune cell derived cytokines.

Embodiment 11. The composition according to embodiments 1-6, wherein said bacterial strains decrease the production of Th17 and immune cell derived cytokines.

Embodiment 12. The composition according to embodiment 10, wherein the Th2 and immune cell derived cytokines are IL-5 and IL-13.

Embodiment 13. The composition according to embodiment 11, wherein the Th17 and immune cell derived cytokines are IL-17 and IL-22.

Embodiment 14. The composition according to any one of embodiments 1 to 13, wherein said composition is a food product, a food ingredient, a dietary supplement or a pharmaceutically acceptable composition.

Embodiment 15. The composition according to embodiment 14, wherein the pharmaceutical composition is in the form of creams or sprays, such as nasal sprays.

Embodiment 16. Use of a composition comprising an effective amount of one or more bacterial strains selected from the species Lactobacillus acidophilus and/or Lacticaseibacillus paracasei for the modulation of an allergic immune response in a subject.

Embodiment 17. The use according to embodiment 16, wherein said composition comprises bacterial strains from the species Lactobacillus acidophilus and from the species Lacticaseibacillus paracasei.

Embodiment 18. The use according to embodiments 16 or 17, wherein the bacterial strain of the species Lactobacillus acidophilus is strain La-14.

Embodiment 19. The use according to embodiment 16 or 17, wherein the bacterial strain of the species Lacticaseibacillus paracasei is strain Lpc-37.

Embodiment 20. The use according to any one of embodiments 16-19, wherein the bacterial strains are present in at least 1×10⁶ colony forming units (CFUs) per dose, preferably between 1×10⁶ and 1×10¹⁴ and more preferably between 1×10⁸ and 1×10¹².

Embodiment 21. The use according to any one of embodiments 16-20, wherein said modulation of the allergic immune response alleviates symptoms of allergic diseases.

Embodiment 22. The use according to embodiment 21, wherein the allergic disease is allergic rhinitis.

Embodiment 23. The use according to embodiment 21, wherein the allergic disease is atopic eczema, allergic asthma, food allergies, contact dermatitis, drug and insect bite anaphylaxis.

Embodiment 24. The use according to embodiments 16-20, wherein said bacterial strains decrease the production of Th2 and immune cell derived cytokines.

Embodiment 25. The use according to embodiments 16-20, wherein said bacterial strains decrease the production of Th17 and immune cell derived cytokines.

Embodiment 26. The use according to embodiment 24, wherein the Th2 and immune cell derived cytokines are IL-5 and IL-13.

Embodiment 27. The use according to embodiment 25, wherein the Th17 and immune cell derived cytokines are IL-17 and IL-22.

Embodiment 28. The use according to any one of embodiment 16 to 27, wherein said composition is a food product, a food ingredient, a dietary supplement or a pharmaceutically acceptable composition.

Embodiment 29. The according to embodiment 28, wherein the pharmaceutical composition is in the form of creams or sprays, such as nasal sprays.

Embodiment 30. A method of modulation of an allergic immune response in a subject in need thereof, said method comprising administering to said subject a composition comprising an effective amount of one or more bacterial strains selected from the species Lactobacillus acidophilus and/or Lacticaseibacillus paracasei.

Embodiment 31. The method according to embodiment 30, wherein said composition comprises bacterial strains from the species Lactobacillus acidophilus and from the species Lacticaseibacillus paracasei.

Embodiment 32. The method according to embodiment 30 or 31, wherein the bacterial strain of the species Lactobacillus acidophilus is strain La-14.

Embodiment 33. The method according to embodiment 30 or 31, wherein the bacterial strain of the species Lacticaseibacillus paracasei is strain Lpc-37.

Embodiment 34. The method according to any one of embodiments 30-33, wherein the bacterial strains are present in at least 1×10⁶ colony forming units (CFUs) per dose, preferably between 1×10⁶ and 1×10¹⁴ and more preferably between 1×10⁸ and 1×10¹².

Embodiment 35. The method according to any one of embodiment 30-34, wherein said modulation of the allergic immune response alleviates symptoms of allergic diseases.

Embodiment 36. The method according to embodiment 35, wherein the allergic disease is allergic rhinitis.

Embodiment 37. The method according to embodiment 36, wherein the allergic disease is atopic eczema, allergic asthma, food allergies, contact dermatitis, drug and insect bite anaphylaxis.

Embodiment 38. The composition according to embodiment 30-34, wherein said bacterial strains decrease the production of Th2 and immune cell derived cytokines.

Embodiment 39. The method according to embodiment 30-34, wherein said bacterial strains decrease the production of Th17 and immune cell derived cytokines.

Embodiment 40. The composition according to embodiment 38, wherein the Th2 and immune cell derived cytokines are IL-5 and IL-13.

Embodiment 41. The composition according to embodiment 39, wherein the Th17 and immune cell derived cytokines are IL-17 and IL-22.

Embodiment 42. The method according to any one of embodiment 30 to 41, wherein said composition is a food product, a food ingredient, a dietary supplement or a pharmaceutically acceptable composition.

Embodiment 43. The method according to embodiment 42, wherein the pharmaceutical composition is in the form of creams or sprays, such as nasal sprays.

### EXAMPLES

The following examples are provided to demonstrate and further illustrate specific embodiments and aspects of the present invention and are not to be construed as limiting the scope thereof.

### Materials And Methods

### Strains and Growth Conditions

The effect of Lactobacillus acidophilus La-14 (DGCC 11491) and Lacticaseibacillus paracasei Lpc-37 (DGCC 4981) on Th2 and Th17 cell responses of human peripheral blood mononuclear cells (PBMCs) stimulated by phytohemagglutinin (PHA) was evaluated. PBMCs are a general ex vivo model stimulated with T cell mitogen PHA for assessing allergic responses. PBMCs were separated from buffy coats purchased from Finnish Red Cross Blood Service, under Helsinki and Uusimaa Hospital District ethical committee permission (Approval: HUS/828/2020). Buffy coats were diluted 1:1 in 2 mM EDTA (Gibco) in phosphate buffered saline (PBS, Thermo Fisher Scientific) and PBMCs were isolated by Ficoll-Paque (GE Healthcare) density gradient centrifugation using SepMate tubes (Stemcell technologies, Grenoble, France). After washing cells with PBS (Thermo Fisher Scientific), the cells were calculated and diluted in cell culture medium RPMI 1640 (Merck) supplemented with 10% fetal bovine serum and 1% Antibiotic-Antimycotic (both from Thermo Fisher Scientific). The cells were plated on non-tissue treated 24 well plates (Falcon) 10\hat{ }6 cell/well.

Probiotic bacterial strains were used in the experiments from overnight subcultures. Probiotics were collected by centrifugation, washed once with PBS (Thermo Fisher Scientific), and suspended into cell culture medium RPMI 1640. The OD600 was adjusted to correspond to a predetermined bacteria:host cell ratio 10:1. The number of bacteria was calculated before the experiments by flow cytometry and a trend line was established of the growth curve OD values for the strain. This trendline enabled the calculation of bacterial counts based on the OD value without having to calculate the amount of the bacteria separately in each experiment.

Phytohemagglutinin L (PHA-L, Merck) (“PHA”), was dissolved in RPMI 1640 (without serum and antibiotics) and stored in aliquots at −20° C. PHA-L was used in concentration of 1 μg/ml. The PBMCs were treated with individual probiotic strains or combination of two strains with PHA challenge in three replicate wells. For baseline control, PBMCs were treated with PHA challenge without probiotics. Supernatant samples for ELISA testing were collected after 24-hour incubation and stored at −80° C. until analyzed.

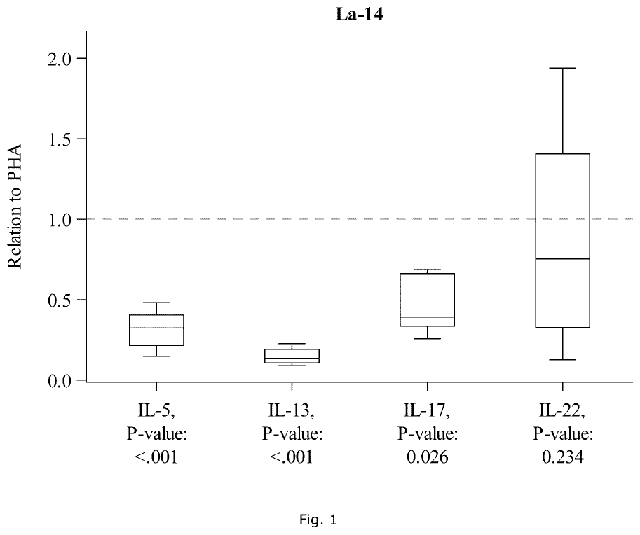

Cell culture supernatants from PBMC cell cultures were analyzed for IL-5, IL-13, IL-17A and IL-22 cytokines with human ELISA kit (Quanterix, Billerica, MA, USA). Results were analyzed with Quanterix SP-X Analysis Software, and graphically illustrated by boxplots (FIGS. 1, 2 and 3). Boxplots were drawn for mean values of the three replicate wells from four PBMC donors normalized by the PHA control samples.

### Statistical Analysis

The mean values of the three replicate wells were used in the analysis. The primary efficacy variable for experiments was the fold-change respective to PHA control in each cytokine, which was calculated for each probiotic treatment as the measured cytokine value divided by the mean value of the same donor's three PHA control values. Differences against PHA control were evaluated by using repeated measurements of analysis of variance method (RM-ANOVA) with probiotic as main effect and donor as random effect in the model. Comparison to PHA control for the fold changes was done in the same analysis model with contrasts by comparing the probiotic estimate value to 1 (resembling no difference). P-values and 95% confidence intervals were estimated for all comparisons.

### Results

The effect of strain La-14 on PHA induced cytokine expression in PBMCs was tested. The cells were treated with PHA alone or in combination with La-14. The cytokine concentrations were measured from the supernatants and the results show statistically significant decrease in IL-5, IL-13, and IL-17 response by strain La-14 compared with the PHA stimulation alone (FIG. 1).

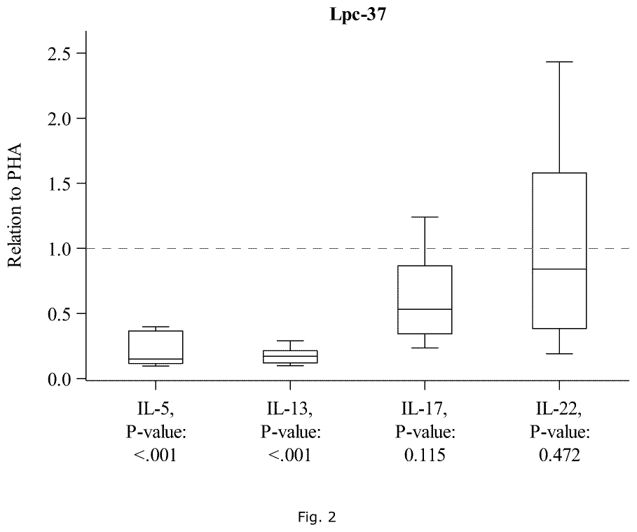

The effect of strain Lpc-37 on PHA induced cytokine expression in PBMCs was tested. The cells were treated with PHA alone or in combination with Lpc-37. The cytokine concentrations were measured from the supernatants and the results show statistically significant decrease in IL-5 and IL-13 response by strain Lpc-37vcompared with the PHA stimulation alone (FIG. 2).

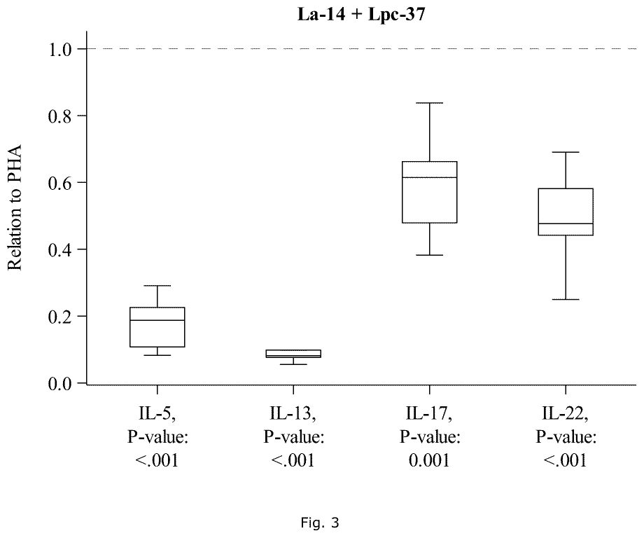

The effect of strains La-14 and Lpc-37 combined on PHA induced cytokine expression in PBMCs was tested. The cells were treated with PHA alone or in combination with strains La-14 and Lpc-37 combined. The cytokine concentrations were measured from the supernatants and the results show statistically significant decrease in IL-5, IL-13, IL-17, and IL-22 response by the combination of strains La-14 and Lpc-37 compared with the PHA stimulation alone (FIG. 3).

The present invention shows that Lactobacillus acidophilus La-14, Lacticaseibacillus paracasei Lpc-37, and their combination, decrease the expression of Th2 and Th17 cytokines (IL-5, IL-13, IL-17, and IL-22) in PHA stimulated PBMCs (FIG. 3). The expression of IL-17 was not decreased by Lpc-37 alone and IL-22 cytokine is not decreased by La-14 or Lpc-37 alone (FIGS. 1 and 2), however the combination of La-14 and Lpc-37 is able to reduce IL-17 and IL-22 significantly which shows the synergistic effect of these strains (FIG. 3).

The present results indicate that the probiotics La-14 and Lpc-37 are able to downregulate the allergen-specific immune responses, and thus prevent or alleviate symptoms of allergic diseases such as allergic rhinitis, food allergies, atopic dermatitis, and allergic asthma.

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. A therapeutic composition comprising:
one or more bacterial strains selected from the species Lactobacillus acidophilus and/or Lacticaseibacillus paracasei in an amount effective for use in the modulation of an allergic immune response in a subject; and
a pharmaceutically acceptable carrier, a pharmaceutically acceptable diluent, a pharmaceutically acceptable excipient or a pharmaceutically acceptable adjuvant.

2. (canceled)

3. The composition according to claim 1, wherein said composition comprises bacterial strains from the species Lactobacillus acidophilus and from the species Lacticaseibacillus paracasei.

4. The composition according to claim 1, wherein the composition comprises strain La-14 or Lpc-37.

5-29. (canceled)

30. A method of modulation of an allergic immune response in a subject in need thereof, said method comprising administering to said subject a composition comprising an effective amount of one or more bacterial strains selected from the species Lactobacillus acidophilus and/or Lacticaseibacillus paracasei.

31. The method according to claim 30, wherein said composition comprises bacterial strains from the species Lactobacillus acidophilus and from the species Lacticaseibacillus paracasei.

32. The method according to claim 30, wherein the composition comprises strain La-14.

33. The method according to claim 30, wherein the composition comprises strain Lpc-37.

34. The method according to claim 30, wherein the bacterial strain(s) is/are present in at least 1×10⁶ colony forming units (CFUs) per dose.

35. The method according to claim 30, wherein said modulation of the allergic immune response alleviates symptoms of allergic diseases.

36. The method according to claim 35, wherein the allergic disease is allergic rhinitis.

37. The method according to claim 36, wherein the allergic disease is selected from atopic eczema, allergic asthma, food allergies, contact dermatitis, drug and insect bite anaphylaxis.

38. The composition according to claim 30, wherein said bacterial strain(s) decrease(s) the production of Th2 and immune cell derived cytokines.

39. The method according to claim 30, wherein said bacterial strain(s) decrease(s) the production of Th17 and immune cell derived cytokines.

40. The composition according to claim 38, wherein the Th2 and immune cell derived cytokines are IL-5 and IL-13.

41. The composition according to claim 39, wherein the Th17 and immune cell derived cytokines are IL-17 and IL-22.

42. The method according to claims 30, wherein said composition is a food product, a food ingredient, a dietary supplement or a pharmaceutically acceptable composition.

43. The method according to claim 42, wherein the pharmaceutical composition is in the form of a cream or spray.

44. The composition according to claim 1, wherein the composition comprises a dietary supplement or pharmaceutically acceptable composition.

45. The composition according to claim 1, wherein the composition comprises a pharmaceutical composition in the form of a cream or spray.

46. The composition according to claim 1, wherein the composition comprises a pharmaceutical composition in the form of a nasal spray.
